(19) United States
(12) Patent Application Publication
(10) Pub. No.: US 2026/0224675 A1
(43) Pub. Date: Aug. 6, 2026

(54) METHOD OF INHIBITING DEGRADING PROTEASE ACTIVITY IN THE SKIN

(71) Applicant: The Regents of the University of California, Oakland, CA (US)

(72) Inventors: Geert W. Schmid-Schoenbein, Del Mar, CA (US); Frank A. DeLano, San Diego, CA (US)

(21) Appl. No.: 19/149,294

(22) PCT Filed: Jul. 7, 2023
(86) PCT No.: PCT/US2023/027104

§ 371 Date: Jul. 18, 2025

Related U.S. Application Data
(60) Provisional application No. 63/480,820, filed on Jan. 20, 2023.

(51) Int. Cl. A61K 38/57; A61K 31/145; A61K 31/195; A61K 31/245; A61K 31/662; A61K 38/12; A61K 38/48; A61P 17/00

(52) U.S. Cl. CPC A61K 38/57; A61K 31/145; A61K 31/195; A61K 31/245; A61K 31/662; A61K 38/12; A61K 38/482; A61P 17/00; C12Y 304/21078; C12Y 304/21079

(57) ABSTRACT

Provided herein are methods of reversing accumulation of a pancreatic digestive serine protease, reversing cellular damage, and/or preserving the extracellular matrix in skin tissue of a subject comprising selecting a subject at risk of skin tissue damage, and administering a therapeutically effective amount of a serine protease inhibitor. The serine protease inhibitor may be an orally administered inhibitor or the serine protease inhibitor may be applied directly to the skin.

### CROSS-REFERENCE TO RELATED APPLICATIONS

This application claims priority to U.S. Provisional Patent Application No. 63/480,820, filed on Jan. 20, 2023. The disclosure of the prior application is considered part of the disclosure of this application, and is incorporated herein by reference in its entirety.

### BACKGROUND

Proposed fundamental mechanisms (as compared to specific cell dysfunctions) of aging include: radiation exposure, oxygen free radical damage, and accumulation of “micro injuries” over time. Caloric restriction (while maintaining basic nutritional support, such as essential amino acids, vitamins and minerals) is the only intervention known to increase lifetime and decrease many comorbidities (e.g., metabolic diseases, chronic degenerative diseases, several forms of cancer, and others). Chronic matrix metalloproteinase (MMP) inhibition has also been suggested to reduce symptoms of the metabolic syndrome (e.g., grape seed extract, resveratrol, and others) in experimental models and short term human trials. Multiple genes have been identified in specific aging human populations and animal models that are associated (e.g., by genome wide association studies) with longevity. However, each of these theories is incomplete.

### SUMMARY

Provided herein are methods of reversing accumulation and activity of pancreatic serine proteases in skin tissue of a subject, including (a) selecting a subject having or at risk of accumulation of pancreatic serine proteases in the skin tissue; and (b) administering a therapeutically effective amount of a serine protease inhibitor, thereby reversing accumulation of and activity of the pancreatic serine proteases in the skin tissue of the subject.

Also provided herein are methods of reversing cellular damage in skin tissue of a subject, including (a) selecting a subject having or at risk of cellular damage to the skin tissue; and (b) administering a therapeutically effective amount of a pancreatic serine protease inhibitor, thereby reversing cellular damage in the skin tissue of the subject.

Also provided herein are methods of preserving extracellular matrix in the skin tissue of a subject, including (a) selecting a subject having or at risk of damage to the extracellular matrix in the skin tissue; and (b) administering a therapeutically effective amount of a pancreatic serine protease inhibitor, thereby preserving extracellular matrix in the skin tissue of the subject.

In some embodiments, the subject is at least 40 years old. In some embodiments, the subject is at least 50 years old. In some embodiments, the subject is at least 60 years old. In some embodiments, the subject is not at risk of developing shock and/or septic shock. In some embodiments, the subject does not have HIV.

In some embodiments, the pancreatic serine protease comprises at least one of a trypsin, a subtilisin, or combinations thereof. In some embodiments, the pancreatic serine protease comprises at least one of a trypsin, an elastase, a chymotrypsin, or combinations thereof. In some embodiments, the pancreatic serine protease comprises a trypsin.

In some embodiments, the serine protease inhibitor is a competitive inhibitor. In some embodiments, the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soybean trypsin inhibitor, meprin inhibitors, setmelanotide, alpha-1-antitrypsin, and serpin. In some embodiments, the serine protease inhibitor comprises FOY. In some embodiments, the serine protease inhibitor comprises Futhane. In some embodiments, the serine protease inhibitor comprises tranexamic acid.

In some embodiments, the therapeutically effective amount of the serine protease inhibitor is less than 10% of the subject's digestive enzyme activity. In some embodiments, the therapeutically effective amount of the serine protease inhibitor is less than 10 μM. In some embodiments, the therapeutically effective amount of the serine protease inhibitor is less than 5 μM.

In some embodiments, the serine protease inhibitor is orally administered. In some embodiments, the serine protease inhibitor is administered as a liposomal encapsulation. In some embodiments, the serine protease inhibitor is administered by a slow release device. In some embodiments, the serine protease inhibitor comprises a composition that can be applied to the skin tissue. In some embodiments, the serine protease inhibitor is administered superficially onto the skin tissue.

In some embodiments, the serine protease inhibitor is administered for more than 1 week. In some embodiments, the serine protease inhibitor is administered for more than 2 weeks. In some embodiments, the serine protease inhibitor is administered for more than 4 weeks.

Also provided herein are pharmaceutical compositions for the treatment of aging or age-related conditions of skin tissue comprising a serine protease inhibitor.

In some embodiments, the serine protease inhibitor is a competitive inhibitor. In some embodiments, the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soybean trypsin inhibitor, meprin inhibitors, setmelanotide, alpha-1-antitrypsin, and serpin. In some embodiments, the serine protease inhibitor comprises FOY. In some embodiments, the serine protease inhibitor comprises Futhane. In some embodiments, the serine protease inhibitor comprises tranexamic acid.

In some embodiments, the serine protease inhibitor is administered at less than 10% of the subject's digestive enzyme activity. In some embodiments, the serine protease inhibitor is less than 10 μM. In some embodiments, the serine protease inhibitor is less than 5 μM.

In some embodiments, the serine protease inhibitor is orally administered. In some embodiments, the serine protease inhibitor is administered as a liposomal encapsulation. In some embodiments, the serine protease inhibitor is administered by a slow release device. In some embodiments, the serine protease inhibitor comprises a composition that can be applied to the skin tissue. In some embodiments, the serine protease inhibitor is administered superficially onto the skin tissue.

In some embodiments, the serine protease inhibitor is administered for more than 1 week. In some embodiments, the serine protease inhibitor is administered for more than 2 weeks. In some embodiments, the serine protease inhibitor is administered for more than 4 weeks.

### BRIEF DESCRIPTION OF DRAWINGS

FIG. 1A shows en-face view of the inner lining of rat small intestine in the jejunum labeled for mucin showing individual intestinal villi for a young (18 week), an old, non-treated (104 weeks), and an old-treated (104 week) animal with the oral serine protease inhibitor tranexamic acid for two weeks. The images show loss of the mucin coat in the small intestine of the old, non-treated rat, and restoration of the mucin coat in the small intestine of the old-treated rat after two weeks of oral treatment with a trypsin inhibitor. The images on the left were recorded during optical focus on the tip of the villi and on the right with focus on the base of the villi, showing reduction of the mucin coat in both layers.

FIG. 1B shows a bar-graph showing mucin optical density measurements (in digital units) at the tip of the villi.

FIG. 2A shows en-face view of pancreatic trypsin (left two rows) and amylase (right two rows) (brown) and co-labeled for mucin by alcian blue in the rat small intestine. Age groups are the same as in FIGS. 1A-1B. The B/W images represent trypsin/amylase label densities after digital color extractions.

FIGS. 2B-2C are bar graphs showing mean±SD for light intensity (digital units between 1 and 255).

FIG. 3A shows skin tissue cross-section in young (Y, 18 week), old (O, 104 week) and old treated (OT, 104 week) rat labeled for pancreatic trypsin.

FIG. 3B is a bar graph showing trypsin intensity (digital units).

FIG. 4A shows collagen fragmentation in the rat skin tissue as detected by collagen hybridizing peptides (CHP). The CHP density after color extraction (bottom row) and digital intensity measurement (bar graph) show the same age groups as in FIGS. 3A-3B. Note the reduction of pancreatic trypsin label density and reduction of collagen fragmentation in the old-treated skin sections.

FIG. 4B is a bar graph showing collagen fragmentation intensity (digital units).

### DETAILED DESCRIPTION

Digestive enzyme activity in organs outside the GI tract has been discovered as a mechanism for chronic and gradual loss of cell and organ dysfunctions during aging. It is an alternative explanation for mechanisms of biological aging to existing theories. The present disclosure describes methods of inhibiting a pancreatic serine protease and decreasing the activity of the pancreatic serine protease outside a gastrointestinal (GI) tract, specifically in the skin tissue of a subject.

Various non-limiting aspects of these methods are described herein, and can be used in any combination without limitation. Additional aspects of various components of the methods described herein are known in the art.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value. As used herein, a “cell” can refer to either a prokaryotic or eukaryotic cell, optionally obtained from a subject or a commercially available source.

### Aging Process

As used herein, the term “aging” refers to the process associated with becoming older. While the term refers especially to human beings, many animals, and fungi, in the broader sense, aging can also refer to single cells within an organism which have ceased dividing (cellular senescence), show reduced cell functions (response to for example growth hormones, insulin) and gene expression. In humans, aging represents the accumulation of changes in tissue properties over time, encompassing physical and psychological changes.

For example, aging is accompanied by a loss of cell and tissue functions, clinically manifesting co-morbidities with increased susceptibility to diseases, and eventual by full organ failure. A spectrum of biological processes (e.g., cell and mitochondrial functions, stem cell proliferation and differentiation, genetic lesions, histones, DNA repair mechanisms, epigenetics, protein folding, intra-and inter-cellular signaling, and nutrient utilization) become dysregulated, unstable, and exhausted. Pathophysiological mechanisms in aging can include impaired resistance to molecular stressors, chronic low-grade inflammation, genomic instability, telomere attrition and cellular senescence, epigenetic alterations, loss of protein homeostasis (proteostasis), deregulated nutrient sensing, stem cell exhaustion, and/or altered intercellular communication. Vascular and immunological cell functions become impaired with pathological restructuring and development of age-related risk factors and diseases, while different tissues share molecular and cellular mechanisms for micro-and macrovascular pathologies in aging. Aging is also accompanied by chronic low-grade inflammation, and since the inflammatory cascade fundamentally serves tissue repair, a chronic mechanism can exist in aging that causes tissue damage. In all organs, the cells and the extracellular matrix are known to degrade, for which mechanisms have been proposed to be due to reactive oxygen species, radiation exposure, and repeat small injuries.

Aging is among the greatest known risk factors for most human diseases: of the roughly 150,000 people who die each day across the globe, about two thirds die from age-related causes. Aging is associated with changes in dynamic biological, physiological, environmental, psychological, behavioral, and social processes. As used herein, “symptoms of biological aging” can refer to common signs and symptoms of aging that can include, but are not limited to, degradation of the extracellular matrix, immune suppression and increased susceptibility to infection, greater risk of heat stroke or hypothermia, skin thinning and wrinkling, bones break more easily, joint changes, ranging from minor stiffness to severe arthritis, slowed and limited movement, decrease in overall energy, increased fatigue rate, constipation, urinary incontinence, cognitive impairment (e.g., slowing of thought, memory, and thinking), reduced reflexes and coordination, difficulty with balance, decrease in visual acuity, diminished peripheral vision, hearing loss, whitening or graying of hair, loss of smell, and weight loss in part due to loss of muscle tissue.

Pancreatic digestive serine protease activity in organs outside the GI tract has been discovered to serve as a mechanism for chronic and gradual loss of cell and organ functions during aging (e.g., “Autodigestion”). After synthesis in the pancreas, digestive enzymes can be discharged into the small intestine where they degrade large masses of biomolecules. In the small intestine, digestive enzymes are concentrated (e.g., at sub-mM level), fully activated and relatively non-specific to facilitate breakdown of diverse polymeric food sources into lower molecular weight monomeric nutrients. Furthermore, autodigestion of one's own intestine is primarily prevented by compartmentalization of the digestive enzymes in the lumen of the intestine by the mucin/epithelial barrier, and while this barrier is always permeable to small molecular nutrients (e.g., ions, amino acids, or monosaccharides) it generally has a low permeability to larger molecules, such as pancreatic serine proteases.

However, sometimes the mucin/epithelial barrier is compromised due to disease or conditions, and sometimes the mucin/epithelial barrier becomes compromised during aging, as older individuals tend to have weaker mucin/epithelial barriers in the GI tract than young individuals.

The present disclosure provides mechanisms for aging due to autodigestion involving pancreatic serine proteases. The methods of the disclosure block digestive serine proteases outside the gastrointestinal tract (GI) tract, specifically in the skin tissue, with minimal effect on pancreatic serine protease activity inside the GI tract to ameliorate symptoms and diseases of aging due to autodigestion.

### Serine Proteases/serine Protease Inhibitors

Serine proteases are sometimes referred to as serine endopeptidases, which are enzymes that can cleave peptide bonds in proteins. There are two main categories of serine proteases based on their structure: chymotrypsin-like (trypsin-like) and subtilisin-like.

Subtilisin-like serine proteases can be found in prokaryotes and share the same catalytic mechanism as the trypsin-like serine proteases. The chymotrypsin-like/trypsin-like serine proteases contain two beta-barrel domains that converge at a catalytic site. Serine proteases are folded in such a way that they utilize a catalytic triad located in the active site of the enzyme, which consists of three amino acids, Histidine 57, Serine 195, and Aspartic acid 102. Additionally, elastase is a serine protease produced by the pancreas that catalyzes cleavage of carboxyl groups present on small hydrophobic amino acids, such as glycine, alanine, and valine. The primary role of elastase is the breakdown of elastin, a protein that imparts elasticity to connective tissue. In some embodiments, a serine protease is a digestive serine protease e.g., a pancreatic serine protease.

Serine proteases can be inhibited by serine protease inhibitors, which can include chemical inhibitors as well as proteinaceous inhibitors. In non-limiting embodiments, small molecular weight inhibitors can pass out of the small intestine and into blood, plasma, or other tissues. Sometimes serine protease inhibitors are called SERPINs. Serine protease inhibitors can include competitive inhibitors, non-competitive inhibitors, permeant inhibitors, reversible inhibitors, and irreversible inhibitors. Sometimes serine protease inhibitors block a serine protease by changing the conformational shape of the serine protease, disrupting the active site of the serine protease. Sometimes serine protease inhibitors bind to and block the active site of a serine protease.

Non-limiting examples of serine protease inhibitors include Lepirudin, Bivalirudin, Argatroban, Chymostatin, Benzamidine, Ximelagatran, Rivaroxaban, Idraparinux, Apixaban, Otamixaban, Aprotinin, Dabigatran etexilate, Edoxaban, Letaxaban, Ulinastatin, Darexaban, Nafamostat, Gabexate, Sivelestat, Melagatran, Cholesterol sulfate, Dabigatran, Fondaparinux, Desirudin, Betrixaban, CGS-27023, GW-813893, Berotralstat, Evolocumab, Conestat alfa, Rosmarinic acid, Alpha-1 antitrypsin, Alpha-2 antiplasmin, BIA 10-2472, C1-inhibitor, Camostat, Cospin, CU-2010, CU-2020, Kallistatin, Kazal domain, Maspin, Methoxy arachidonyl fluorophosphonte, Microviridin, Plasminogen activator inhibitor-1, Plasminogen activator inhibitor-2, PMSF, Protein C inhibitor, Protein Z-related protease inhibitor, SERPINA9, SERPINB1, SERPINB3, SERPINB4, SERPINB6, SERPINB7, SERPINB8, SERPINB9, SERPINB13, SERPINE2, SPINT1, Spaostat, and Uterine Serpin.

In some embodiments, the serine protease inhibitor of the methods of the disclosure includes nafamostat mesylate (Futhan), camostat mesilate (FOY 305), gabexate mesilate (FOY) or derivatives, serine protease inhibitor Kazal-type 1 (SPINK1), tranexamic acids, granzyme A, granzyme B, UAMC-00050, 4-(2-minoethyl) benzenesulfonyl fluoride hydrochloride (AEBSF), soybean trypsin inhibitor, meprin inhibitors, setmelanotide, or alpha-1-antitrypsin. In some embodiments, the serine protease inhibitor can include a derivative of any one of the serine protease inhibitors described herein.

### Pharmaceutical Compositions for the Treatment of Age-related Conditions

The methods described herein include the use of pharmaceutical compositions comprising one or more of serine protease inhibitors as an active ingredient.

As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Pharmaceutical compositions are typically formulated to be compatible with its intended route of administration. Examples of routes of administration include parenteral, e.g., intravenous, subcutaneous, oral (e.g., capsules or inhalation), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a pharmaceutical composition as described herein can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

The pharmaceutical compositions can also be prepared in the form of suppositories (e.g., with conventional suppository bases such as cocoa butter and other glycerides) or retention enemas for rectal delivery.

In one embodiment, the pharmaceutical compositions are prepared with carriers that will protect the pharmaceutical compositions against rapid elimination from the body, such as a controlled release formulation, including implants and microencapsulated delivery systems. In some embodiments, the pharmaceutical compositions include a serine protease inhibitor that is linked, conjugated, or fused to another molecule. In some embodiments, the other molecule changes a property of the pharmaceutical composition. In some embodiments, pharmaceutical compositions can be delivered by using nanoparticle encapsulation. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration. The pharmaceutical compositions can be over-the-counter compositions.

The pharmaceutical compositions can be cosmetic compositions. In some embodiments, the pharmaceutical compositions can be topical compositions. In some embodiments, the pharmaceutical compositions can be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (OMI) or conversely (W/O); or suspensions or emulsions of smooth, semi-solid or solid consistency of the cream or gel type.

In some embodiments, the pharmaceutical compositions can also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the field, and, for example, range from 0.01% to 10% of the total weight of the composition. In some embodiments, depending on their nature, these additives and adjuvants may be introduced into the fatty phase, or into the aqueous phase.

In some embodiments, the pharmaceutical compositions can optionally comprise other skin benefit materials. These include estradiol; progesterone; pregnanalone; coenzyme Q10; methylsolanomethane (MSM); copper peptide (copper extract); plankton extract (phytosome); glycolic acid; kojic acid; ascorbyl palmitate; all-trans-retinol; azaleic acid; salicylic acid; broparoestrol; estrone; adrostenedione; androstanediols; and any combinations thereof.

### Methods of Decreasing Pancreatic Serine Protease Activity Outside of the Gastrointestinal (GI) Tract

Aging and/or age-related diseases or conditions can cause an increase in the permeability of the intestinal barrier to digestive pancreatic serine proteases such that pancreatic serine protease activity may be detectable in the circulation of the subject. Digestive enzymes can leak across the mucin-epithelial barrier into tissues and organs outside the pancreas and intestines where they may damage the extracellular matrix and cell membranes. In some embodiments, damage may include ectodomain receptor cleavage.

The digestive enzymes can cause multiple forms of tissue damage, including cleavage of membrane receptors (e.g., the insulin receptor, growth hormone receptor) and degradation of collagen in the skin of a subject. Pancreatic trypsin can also activate prohormones and interfere with physiological signaling due to its ability to cleave a broad spectrum of humoral mediators as well as their receptors. Treatment with administration of a digestive enzyme inhibitor (e.g., serine protease inhibitor, e.g., trypsin inhibitor) can attenuate breakdown of the mucin barrier, reduce the accumulation of digestive enzymes in the skin, as well as cleavage of collagen. For example, interventions against pancreatic trypsin outside the small intestine not only block activation of secondary proteases (e.g., proMMPs), but also maintain a spectrum of cell functions (including, but not limited to, immune responses, mitochondrial functions, stem cell proliferation and differentiation, DNA repair mechanisms, epigenetics, protein folding, intra-and inter-cellular signaling, and nutrient utilization).

The compositions described herein can be administered to a subject to treat or prevent diseases, disorders, or conditions described herein. In some embodiments, the present disclosure describes methods of reversing accumulation of a pancreatic serine protease in the skin of a subject, reversing cellular damage in the skin of a subject, and/or preserving extracellular matrix in the skin of a subject, by selecting a subject at risk of damage to the skin, and administering a therapeutically effective amount of a serine protease inhibitor.

In some embodiments, the present disclosure describes methods of decreasing pancreatic serine protease activity outside a gastrointestinal (GI) tract of a subject for the purpose of treating age-related conditions in the skin of the subject. In some embodiments, the methods can inhibit or reduce activity of a pancreatic serine protease outside a gastrointestinal (GI) tract of a subject, or reduce symptoms of biological aging in a subject. In some embodiments, the methods include administering to a subject in need thereof a therapeutically effective amount of a serine protease inhibitor that results in the decrease in the activity of the digestive pancreatic serine protease outside the GI tract.

In some embodiments, the methods include prophylactically treating age-related diseases or conditions of the skin. As used herein, the term “prophylactically treating” can refer to taking preventative measures to preserve health or prevent the progression of or occurrence of a disease or condition (e.g., reversing accumulation of a digestive pancreatic serine protease in the skin of a subject, reversing cellular damage in the skin of a subject, and/or preserving extracellular matrix in the skin of a subject). For example, a subject can be prophylactically treated when the subject is at risk of experiencing a disease or condition (e.g., having biomarkers that increase susceptibility of a particular condition, e.g., wrinkles, psoriasis, rosacea, melasma).

### Subject

As used herein, the term “subject” refers to an organism, typically a mammal (e.g., a human). In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

In some embodiments, the subject can be an animal, human or non-human. Non-limiting examples of non-human subjects can include mice, rats, hamsters, rabbits, cats, dogs, horses, pigs, donkeys, monkeys, and/or other non-human primates such as apes and lemurs.

In some embodiments, the subject is a human. In some embodiments, a human patient can be an adult human or juvenile human (e.g., human below the age of 18 years old). In some embodiments, the subject is a patient suffering from an age-related disease, disorder, or condition. In some embodiments, the subject is a patient susceptible to an age-related disease, disorder, or condition. In some embodiments, the subject is a patient displaying one or more signs or symptoms or characteristics of an age-related disease, disorder, or condition. In some embodiments, the subject is displaying symptoms of an age-related disease, disorder, or condition when the subject is considered biologically aged, e.g., over 50 years old, over 55 years old, over 60 years old, over 65 years old, over 70 years old, over 75 years old, over 80 years old, over 85 years old, over 90 years old, or over 95 years old. In some embodiments, the subject is displaying symptoms of an age-related disease, disorder, or condition at time when the subject is not considered biologically aged, e.g., under 45 years old, under 40 years old, under 35 years old, under 30 years old, or under 25 years old. In some embodiments, the subject is an adult human over the age of 18 years old. In some embodiments, the subject is older than 20 years old. In some embodiments, the subject is older than 30 years old. In some embodiments, the subject is older than 40 years old. In some embodiments, the subject is older than 50 years old. In some embodiments, the subject is older than 60 years old. In some embodiments, the subject is older than 70 years old. In some embodiments, the subject is older than 80 years old. In some embodiments, the subject is older than 90 years old. In some embodiments, the subject is older than 100 years old.

### Treating/treatment

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition.

Those of ordinary skill in the art will appreciate that the term “therapeutically effective amount” does not in fact require successful treatment be achieved in a particular individual.

Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse aging-supportive process occurring in said individual, or will enhance or increase an aging-suppressive process in said individual. A “therapeutically effective amount” of a composition described herein can reverse (in a therapeutic treatment) the development accumulation of a pancreatic serine protease in an organ of a subject, reverse cellular damage in an organ of a subject, or preserve extracellular matrix structure in an organ of a subject. A therapeutically effective amount can include preserving the molecular structure of organ tissue as detected by hybridizing peptides that can bind to collagen structure at cleavage sites. A therapeutically effective amount administered to an individual to treat a disease or condition in that individual may be the same or different from a therapeutically effective amount administered for prophylactic purposes. The therapeutic methods described herein are not to be interpreted as, restricted to, or otherwise limited to a “cure” for aging; rather the methods of treatment are directed to the use of the described compositions to “treat” age-related conditions, i.e., to effect a desirable or beneficial change in the health of an individual who has an age-related condition, such as but not limited to accumulation of a pancreatic serine protease in an organ of a subject, reverse and/or stop ongoing extracellular matrix protein (e.g., collagen) cleavage, cellular damage (e.g., membrane receptor cleavage) and cellular dysfunction (e.g., reduced integrin attachment to the extracellular matrix and intracellular integrin signaling) in an organ of a subject, or preserve extracellular matrix in an organ of a subject. As is understood in the art, an effective amount of a serine protease inhibitor may vary, depending on, inter alia, patient history as well as other factors such as the type (and/or dosage) of serine protease inhibitor used.

The phrases “reduced”, “decreased”, “a reduced level”, or “a decreased level” and similar phrases generally refer to a reduction or decrease of at least 1% (e.g., at least 2%, at least 4%, at least 6%, at least 8%, at least 10%, at least 12%, at least 14%, at least 16%, at least 18%, at least 20%, at least 22%, at least 24%, at least 26%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference level or value. The phrases “increased”, “greater”, “an increased level”, or “a greater level” and similar phrases generally refer to an increase of at least 1% (e.g., at least 2%, at least 4%, at least 6%, at least 8%, at least 10%, at least 12%, at least 14%, at least 16%, at least 18%, at least 20%, at least 22%, at least 24%, at least 26%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, 100%, 150%, 200%, or more) as compared to a reference level or value.

In some embodiments, a therapeutically effective amount may be formulated and/or administered in a plurality of doses, for example, as part of a dosing regimen. In some embodiments, effective amounts and schedules for administering the serine protease inhibitor described herein may be determined empirically, and making such determinations is within the skill in the art. Those skilled in the art will understand that the dosage that must be administered will vary depending on, for example, the subject that will receive the serine protease inhibitor disclosed herein, the subject's comorbidities (e.g., diabetes, infections) which may influence the skin tissue properties, the intestine's leak of digestive proteases, the route of administration, the particular type of serine protease inhibitor, and other drugs being administered to the subject. In some embodiments, the administration of a therapeutically effective amount comprises chronic administration, whereas in other embodiments the administration of a therapeutically effective amount comprises a scheduled administration.

In some embodiments, the scheduled administration includes a predetermined schedule. Non-limiting examples of a scheduled basis include every other day, every two days, every three days, every four days, every five days, every six days, or once a week. Other non-limiting examples of a scheduled basis include one day on: six days off, two days on: five days off, three days on: four days off, four days on: three days off, five days on:

two days off, six days on: one day off. Yet further non-limiting examples of a scheduled basis include two days on: one day off, two days on: two days off, two days on: three days off, two days on-four days off, two days on: five days off, three days on: one day off, three days on: two days off, three days on: three days off, three days on: four days off, four days on: one day off, four days on-two days off, four days on: three days off, five days on: one day off, five days on: two days off, six days on: one day off. Yet further non-limiting examples of a scheduled basis include one day out of every seven days, two days out of every seven days, three days out of every seven days, four days out of every seven days, five days out of every seven days, or six out of every seven days. The method may comprise administering the composition by a weekly protocol consisting of daily administration of a maintenance dose composition for 3-5 consecutive days followed by no administration for 1-3 consecutive days. Yet further non-limited examples of a scheduled basis can include administration of the serine protease inhibitor before a meal, during a meal, after a meal, or any combinations thereof.

In some embodiments, the subject can be administered the serine protease inhibitor over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of aging). A skilled medical professional can measure pancreatic serine protease activity in a subject's biological fluid in order to inform serine protease inhibitor treatment of the current methods (e.g., the components and methods described in U.S. Pat. Nos. 9,733,241; 9,222,119; 8,940,866; 8,507,218). As described herein, a skilled medical professional can also change the identity and number (e.g., increase or decrease) of the serine protease inhibitor administered to the subject and can also adjust (e.g., increase or decrease) the dosage or frequency of administration of the serine protease inhibitor to the subject based on an assessment of the effectiveness of the treatment. In some embodiments, the serine protease inhibitor is administered for more than 1 week. In some embodiments, the serine protease inhibitor is administered for more than 2 weeks. In some embodiments, the serine protease inhibitor is administered for more than 4 weeks. In some embodiments, the serine protease inhibitor is administered for more than one month, more than two months, more than three months, more than four months, more than five months, more than six months, more than seven months, more than eight months, more than nine months, more than 10 months, more than 11 months, more than 12 months, or longer. In some embodiments, the serine protease inhibitor is administered for more than one year, more than two years, more than three years, more than four years, more than five years, more than six years, more than seven years, more than eight years, more than nine years, more than ten years, or longer.

In some embodiments, the serine protease inhibitor can be administered at a concentration that is lower than the serine protease concentration within the GI tract. In some embodiments, the serine protease inhibitor can be administered at a concentration that is less than 10% of the serine protease concentration of the GI tract. In some embodiments, the serine protease inhibitor can be administered at a concentration that is less than 9%, 8%, 7%, 6%, 5%, 4%, 3%, 2%, or 1% of the serine protease concentration of the GI tract. For example, a small molecular weight serine protease competitive inhibitor (e.g., TXA or FOY), is administered at a concentration (e.g., 10 μM) below the serine protease concentration inside the small intestine (e.g., 100 μM), but which matches and/or exceeds the protease concentration in the plasma (e.g., 5 μM). In some embodiments, the serine protease inhibitor administered blocks the activity of serine proteases in the plasma. In some embodiments, the majority of the digestive activity of the small intestine is preserved. In some embodiments, the concentration of the serine protease inhibitor does not interfere or reduce digestion or functional activity of the stomach and/or small intestine. The serine protease concentration within the GI tract can be determined empirically or it may be determined by consultation to a standardized and accepted source of such information.

In some embodiments, the concentration of the serine protease inhibitor to be administered to a subject can be determined by measuring pancreatic serine protease activity in the subject. In some embodiments, the concentration of the serine protease inhibitor to be administered to a subject can be determined by measuring pancreatic serine protease activity in the subject at a specific time point. In some embodiments, the concentration of the serine protease inhibitor to be administered to a subject can be determined by measuring pancreatic serine protease activity outside the GI tract (e.g., in the skin tissue) of the subject. In some embodiments, pancreatic serine protease concentration within the GI tract is determined by mass spectrometry determination of peptide incidence in plasma. For example, a sample of a patient's plasma can be run through a mass spectrometer and proteolysis of the plasma proteins can be determined. In some embodiments, pancreatic serine protease concentration can be determined by receptor cleavage with antibody against extracellular domains using cells harvested from the subject or the subject's plasma or other body fluid (e.g., lymph fluid).

In some embodiments, the serine protease inhibitor can be administered at a concentration that is lower than the serine protease concentration within the GI tract but higher than the serine protease concentration outside the GI tract (e.g., in plasma, or in peripheral tissues). In some embodiments, the serine protease inhibitor can be administered at a concentration that is about the same as the serine protease concentration outside the GI tract. In some embodiments, the serine protease inhibitor can be administered at a concentration that is higher than the serine protease inhibitor concentration outside the GI tract. In some embodiments, the serine protease inhibitor can be administered at a concentration that is at least 60%, at least 65%, at least 70%, at least 75%, at least 80%, at least 85%, at least 90%, at least 95%, or 100% the concentration of serine protease in the subject's plasma and/or tissue.

In some embodiments, the serine protease inhibitor can be administered at a concentration of less than 10 mM, less than 9 mM, less than 8 mM, less than 7 mM, less than 6 mM, less than 5 mM, less than 4 mM, less than 3 mM, less than 2 mM, less than 1 mM, less than 0.5 mM, or less than 0.1 mM.

In some embodiments, the serine protease inhibitor can be administered at a concentration of less than 50 μM (e.g., less than 48 μM, less than 46 μM, less than 44 μM, less than 42 μM, less than 40 μM, less than 38 μM, less than 36 μM, less than 34 μM, less than 32 μM, less than 30 μM, less than 28 μM, less than 26 μM, less than 24 μM, less than 22μM, less than 20 μM, less than 18 μM, less than 16 μM, less than 14 μM, less than 12μM, less than 10 μM, less than 8 μM, less than 6 μM, less than 5 μM, less than 4 μM, less than 3 μM, less than 2 μM, less than 1 μM, less than 0.5 μM, less than 0.25 μM, or less than 0.1μM). In some embodiments, the serine protease inhibitor is administered at a concentration of less than 5 μM. In some embodiments, the serine protease inhibitor is administered at a concentration of less than 1 μM (e.g., less than 0.8 μM, less than 0.6 μM, less than 0.4 μM, less than 0.2 μM, less than 0.1 μM, less than 90 nM, less than 80 nM, less than 70 nM, less than 60 nM, less than 50 nM, less than 40 nM, less than 30 nM, less than 20nM, less than 10 nM, less than 5 nM, less than 3 nM, less than 1 nM, less than 0.8 nM, less than 0.6 nM, less than 0.4 nM, less than 0.2 nM, less than 0.1 nM, less than 90 pM, less than 80 pM, less than 70 pM, less than 60 pM, less than 50 pM, less than 40 pM, less than 30 pM, less than 20 pM, less than 10 pM, less than 5 pM, less than 3 pM, less than 1 pM, less than 0.8 pM, less than 0.6 pM, less than 0.4 pM, less than 0.2 pM, or less than 0.1 pM).

In some embodiments, the serine protease inhibitor can be administered according to the patient's weight. In some embodiments the serine protease inhibitor can be administered anywhere between 0.01 and 1.0 gm/kg/day. In some embodiments, a therapeutically effective amount of serine protease inhibitor can include 0.01 gm/kg/day, 0.02 gm/kg/day, 0.03 gm/kg/day, 0.04 gm/kg/day, 0.05 gm/kg/day, 0.06 gm/kg/day 0.07 gm/kg/day, 0.08 gm/kg/day, 0.09 gm/kg/day, 0.1 gm/kg/day, 0.11 gm/kg/day, 0.12 gm/kg/day, 0.13 gm/kg/day, 0.14 gm/kg/day, 0.15 gm/kg/day, 0.16 gm/kg/day, 0.17 gm/kg/day, 0.18 gm/kg/day, 0.19 gm/kg/day, 0.20 gm/kg/day, 0.21 gm/kg/day, 0.22 gm/kg/day, 0.23 gm/kg/day, 0.24 gm/kg/day, 0.25 gm/kg/day, 0.26 gm/kg/day, 0.27 gm/kg/day, 0.28 gm/kg/day, 0.29 gm/kg/day, 0.30 gm/kg/day, 0.31 gm/kg/day, 0.32 gm/kg/day, 0.33 gm/kg/day, 0.34 gm/kg/day, 0.35 gm/kg/day, 0.36 gm/kg/day, 0.37 gm/kg/day, 0.38 gm/kg/day, 0.39 gm/kg/day, 0.40 gm/kg/day, 0.41 gm/kg/day, 0.42 gm/kg/day, 0.43 gm/kg/day, 0.44 gm/kg/day, 0.45 gm/kg/day, 0.46 gm/kg/day, 0.47 gm/kg/day, 0.48 gm/kg/day, 0.49 gm/kg/day, 0.50 gm/kg/day, 0.51 gm/kg/day, 0.52 gm/kg/day, 0.53 gm/kg/day, 0.54 gm/kg/day, 0.55 gm/kg/day, 0.56 gm/kg/day, 0.57 gm/kg/day, 0.58 gm/kg/day, 0.59 gm/kg/day, 0.60 gm/kg/day, 0.61 gm/kg/day, 0.62 gm/kg/day, 0.63 gm/kg/day, 0.64 gm/kg/day, 0.65 gm/kg/day, 0.66 gm/kg/day, 0.67 gm/kg/day, 0.68 gm/kg/day, 0.69 gm/kg/day, 0.70 gm/kg/day, 0.71 gm/kg/day, 0.72 gm/kg/day, 0.73 gm/kg/day, 0.74 gm/kg/day, 0.75 gm/kg/day, 0.76 gm/kg/day, 0.77 gm/kg/day, 0.78 gm/kg/day, 0.79 gm/kg/day, 0.80 gm/kg/day, 0.81 gm/kg/day, 0.82 gm/kg/day, 0.83 gm/kg/day, 0.84 gm/kg/day, 0.85 gm/kg/day, 0.86 gm/kg/day, 0.87 gm/kg/day, 0.88 gm/kg/day, 0.89 gm/kg/day, 0.90 gm/kg/day, 0.91 gm/kg/day, 0.92 gm/kg/day, 0.93 gm/kg/day, 0.94 gm/kg/day, 0.95 gm/kg/day, 0.96 gm/kg/day, 0.97 gm/kg/day, 0.98 gm/kg/day, 0.99 gm/kg/day, 1.0 gm/kg/day.

In some embodiments, depending on the properties of the serine protease inhibitor, a therapeutically effective amount of serine protease inhibitor can include amounts higher than 1.0 gm/kg/day. For example, a therapeutically effective amount of serine protease inhibitor can include, 1 gm/kg/day, 2 gm/kg/day, 3 gm/kg/day, 4 gm/kg/day, 5 gm/kg/day, 6 gm/kg/day, 7 gm/kg/day, 8 gm/kg/day, 9 gm/kg/day, 10 gm/kg/day, 11 gm/kg/day, 12 gm/kg/day, 13 gm/kg/day, 14 gm/kg/day, 15 gm/kg/day, 16 gm/kg/day, 17 gm/kg/day, 18 gm/kg/day, 19 gm/kg/day, 20 gm/kg/day, 21 gm/kg/day, 22 gm/kg/day, 23 gm/kg/day, 24 gm/kg/day, 25 gm/kg/day, 26 gm/kg/day, 27 gm/kg/day, 28 gm/kg/day, 29 gm/kg/day, 30 gm/kg/day, 31 gm/kg/day, 32 gm/kg/day, 33 gm/kg/day, 34 gm/kg/day, 35 gm/kg/day, 36 gm/kg/day, 37 gm/kg/day, 38 gm/kg/day, 39 gm/kg/day, 40 gm/kg/day, 41 gm/kg/day, 42 gm/kg/day, 43 gm/kg/day, 44 gm/kg/day, 45 gm/kg/day, 46 gm/kg/day, 47 gm/kg/day, 48 gm/kg/day, 49 gm/kg/day, 50 gm/kg/day, 51 gm/kg/day, 52 gm/kg/day, 53 gm/kg/day, 54 gm/kg/day, 55 gm/kg/day, 56 gm/kg/day, 57 gm/kg/day, 58 gm/kg/day, 59 gm/kg/day, 60 gm/kg/day, 61 gm/kg/day, 62 gm/kg/day, 63 gm/kg/day, 64 gm/kg/day, 65 gm/kg/day, 66 gm/kg/day, 67 gm/kg/day, 68 gm/kg/day, 69 gm/kg/day, 70 gm/kg/day, 71 gm/kg/day, 72 gm/kg/day, 73 gm/kg/day, 74 gm/kg/day, 75 gm/kg/day, 76 gm/kg/day, 77 gm/kg/day, 78 gm/kg/day, 79 gm/kg/day, 80 gm/kg/day, 81 gm/kg/day, 82 gm/kg/day, 83 gm/kg/day, 84 gm/kg/day, 85 gm/kg/day, 86 gm/kg/day, 87 gm/kg/day, 88 gm/kg/day, 89 gm/kg/day, 90 gm/kg/day, 91 gm/kg/day, 92 gm/kg/day, 93 gm/kg/day, 94 gm/kg/day, 95 gm/kg/day, 96 gm/kg/day, 97 gm/kg/day, 98 gm/kg/day, 99 gm/kg/day, or 100 gm/kg/day.

### Administration

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, enteral, parenteral, etc. In some particular embodiments, administration may be bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, topical, tracheal (e.g., by intratracheal instillation), vaginal, vitreal, by patch, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may involve methods of delivery that include, but are not limited to, use of external and/or implanted infusion pumps, liquid formulation, capsulated formulation, or slow release encapsulation.

In some embodiments, the serine protease inhibitor administration can be ocular, oral, parenteral, bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, tracheal (e.g., by intratracheal instillation), vaginal, or vitreal. In some embodiments, the serine protease inhibitor is administered by enteral administration, intraperitoneal administration, intravenous administration, intramuscular administration, subcutaneous administration, intracutaneous administration, oral administration, intranasal administration, intrapulmonary administration, intrarectal administration, or a telemetry controlled external or implanted infusion pump.

In some embodiments, the serine protease inhibitor is administered by oral administration. In some embodiments, the serine protease inhibitor is administered by oral administration comprising drug delivery designs that can minimize small absorption of the serine protease inhibitor (e.g., liposomal encapsulation). In some embodiments, the serine protease inhibitor is administered by subcutaneous delivery into the skin. In some embodiments, the serine protease inhibitor is administered by superficial delivery (e.g., skin lotion). In some embodiments, a concentration of the serine protease inhibitor to be administered to the subject can be determined according to a route of administration. For example, if the serine protease inhibitor is orally administered, the serine protease inhibitor can be administered at a concentration that is lower than the serine protease concentration within the GI tract (e.g., in the lumen of the intestine). However, in some embodiments, if the serine protease inhibitor is administered by transdermal administration, the serine protease inhibitor can be administered at a concentration higher than the serine protease concentration within the GI tract.

In some embodiments, the serine protease inhibitor administration may involve methods of delivery that include, but are not limited to, use of subcutaneous infusion pumps or cutaneous chronic release devices. In some embodiments, a telemetry controlled infusion pump is directed toward the skin tissue or a portion thereof. In some embodiments, the serine protease inhibitor administration can be coupled to feedback of serine protease measurement in the skin. In some embodiments, the serine protease inhibitor can be delivered by transcutaneous injections using a micro-needle and/or arrays of micro-needles.

In some embodiments, the serine protease inhibitor can be administered in a small quantity of a pharmaceutical composition, for example from 1 to 100 ml, is applied to exposed areas of the skin, from a suitable container or applicator and, if necessary, it is then spread over and/or rubbed into the skin using the hand or fingers or a suitable device. In some embodiments, the pharmaceutical composition may be specifically formulated for use as a hand, or as a facial treatment. In some embodiments, the serine protease inhibitor in the pharmaceutical composition can be formulated as a lotion, a cream or a gel. In some embodiments, the composition can be packaged in a suitable container to suit its viscosity and intended use by the subject. In some embodiments, the composition can be applied to exposed areas of the skin from a facial patch or facial mask.

In some embodiments, the subject can be administered more than one serine protease inhibitor. In some embodiments, the administration of the one or more serine protease inhibitors is sequential administration (e.g., one serine protease inhibitor is administered, stopped, and a second, different serine protease inhibitor is administered). In some embodiments, the subject can be administered a combination of serine protease inhibitors at the same time.

In some embodiments, the methods of the disclosure can be administered before, in conjunction with, or after other methods or therapeutic treatments, either for the condition being treated by administration of the serine protease inhibitor, or another condition. In a non-limiting example, a subject can be treated with surgery for a cancerous mass in the intestine, and after the surgery the subject can be administered a serine protease inhibitor to limit leakage of proteases from the intestine. Preventative/prophylactic administration of serine protease inhibitors can be used to slow and/or prevent autodigestion of the subject's skin due to the intestinal permeability, wherein digestive proteases are leaked due to enhanced intestinal permeability.

In some embodiments, pancreatic serine protease activity is increased in a postprandial period. To block this activity, the serine protease inhibitor can be administered before food intake (e.g., eating). In diabetics or pre-diabetics, this postprandial period of elevated pancreatic serine protease activity is longer than in non-diabetics, and may last for several hours. In some embodiments, the amount or concentration of serine protease inhibitor administration will depend on measurements of protease activity in plasma or in peripheral tissues, like abdominal fluid, heart, brain, intestine, kidney, liver, lung, eye, or other tissues disclosed herein. In some embodiments, the serine protease inhibitor is administered during a diurnal cycle, wherein the serine protease inhibitor administration depends on the measured pancreatic serine protease activity in the subject.

### Aging of the Skin Tissue

In some embodiments, the methods provided herein can reduce symptoms of biological aging in a subject. In some embodiments, the subject can display one or more signs or symptoms or characteristics of an age-related disease, disorder, or condition.

In some embodiments, the methods provided herein can reverse accumulation of a pancreatic serine protease in skin tissue of a subject, the method including (a) selecting a subject having or at risk of accumulation of a pancreatic serine protease in the skin tissue; and (b) administering a therapeutically effective amount of a serine protease inhibitor. In some embodiments, the methods provided herein can reverse cellular damage in skin tissue and/or preserve extracellular matrix in the skin tissue, the method including (a) selecting a subject having or at risk of loss of extracellular matrix in the skin tissue; and (b) administering a therapeutically effective amount of a serine protease inhibitor.

In some embodiments, the methods involved selecting a subject at risk of damage to the skin tissue. In some embodiments, the methods provided herein can prevent and/or minimize symptoms of aging of the skin tissue. In some embodiments, symptoms of aging of the skin tissue can include, but are not limited to, thinning of the skin tissue, loss of mechanical strength of the skin tissue, restructuring of follicles and hair growth, and change of skin type (e.g., wrinkling). In some embodiments, damage to the skin tissue can include, but are not limited to, damage to the extracellular matrix (ECM), blood vessels, nerves, lymphatics, connective tissue cells (e.g., fibroblast, mast cells, macrophages, stem cells, or specific immune cells) of the skin tissue. In some embodiments, damage to the skin tissue can also include proteolytic activity (e.g., receptor cleavage) that can result in cell apoptosis. For example, a symptom of aging of the skin tissue can include loss of capillaries (e.g., capillary rarefaction), which can be a result of cleavage of vascular endothelial growth factor (VEGF) receptor and apoptosis of the endothelial cells. In some embodiments, symptoms of aging of the skin tissue can include loss of stem cells in follicles and hair loss. In some embodiments, the methods provided herein can prevent and/or minimize degradation of collagen in the skin tissue. In some embodiments, the pharmaceutical compositions provided herein are for the treatment of wrinkles and fine lines; firming skin tissue; regrowth of the ECM in the dermis and subdermis; thickening of the skin; and reviving the radiance of the skin.

The subject can be at risk of damage to the skin tissue because the subject is exhibiting symptoms consistent with a known disease or condition that affects the skin tissue. The subject can be at risk of damage to the skin tissue because the subject has a biomarker known to predispose the subject to a known disease or condition that affects the skin tissue. The subject can be at risk of damage to the skin because the subject has a family history that would predispose them to a known disease or condition that affects the skin tissue. In some embodiments, the subject can have a chronic metabolic or degenerative disease. In some embodiments, the subject can have a disease or condition that is associated with enhanced digestive enzymes in the skin. In some embodiments, the subject demonstrates symptoms and/or biomarkers of elevated pancreatic serine protease activity outside the GI tract (e.g., in plasma, or in peripheral tissue). For clarity, the methods of the disclosure are not intended to treat shock (e.g., septic shock) or HIV. Further, the methods of the disclosure may further include a step of screening a subject for shock (e.g., septic shock) and/or HIV, and not administering a serine protease inhibitor to the subject if the subject is currently experiencing shock or biomarkers of HIV.

In some embodiments, the aging-related disease or condition can include roughened or dry skin, seborrheic keratoses, cherry angiomas, skin tags, warts, actinic keratosis, age spots, stasis dermatitis, shingles, bed sores, skin tears, rosacea, asteatotic eczema, seborrheic dermatitis, nummular eczema, senile purpura, and/or skin cancers. In some embodiments, the aging-related disease or condition can include thinning of the epidermis, decrease of the number of pigment-containing cells, increase of size of melanocytes, appearance of lentigos, elastosis, solar elastosis, blood vessels of the dermis becoming more fragile, sebaceous glands producing less oil, and/or thinning of the subcutaneous fat layer. In some embodiments, the aging-related disease or condition that affects the skin tissue can include blood vessel diseases (e.g., atherosclerosis), diabetes, heart disease, liver disease, nutritional deficiencies, obesity, bacterial infections, fungal infections, benign and malignant tumors, and/or chronic wounds.

### Kits

Also provided herein are kits for the use in the methods described herein. For example, the kids can include a composition comprising a serine protease inhibitor for topical administration. Instructions for use can also be included in the kits.

### EXAMPLES

### Example 1

### Skin Sample Collection and Pancreatic Serine Protease Inhibition in Rats

### Animals and Tissue Collection

Male Wistar rats (Harlan Sprague Dawley Inc., Indianapolis, IN) at maturity (4 months, 300 to 350 gm) and old age (24 months, 375 to 450 gm) were included in the study. The animals were maintained on standard laboratory chow (8604 Teklad rodent diet; Harlan Laboratories, Indianapolis, IN) without restriction and water ad libitum and maintained in separated room without pathogen-free conditions. They were confirmed to exhibit normal mobility, water and food consumption and fecal material discharge. Animals that exhibited signs of morbidities were excluded. A subgroup of old animals was given over two weeks a pancreatic serine protease (trypsin) inhibitor (tranexamic acid, 14 days) in drinking water (137 mM, exchanged daily) which at a minimum fluid consumption of 40 ml/day amounts to a minimum dose of 0.39 gm/kg/day for 350 gm body weight.

A femoral venous catheter was placed after general anesthesia (pentobarbital sodium, 50 mg/kg [Abbott Laboratories, North Chicago, IL], intramuscularly after local anesthesia with 2% lidocaine HCl [Hospira, Inc, Lake Forrest, IL]). Skin tissue was immediately collected after euthanasia (Beuthanasia i.v., 120 mg/kg, Schering-Plough Animal Health Corp, Union, NJ), fixed (formalin, 10%, neutral buffered, 1 hr), postfixed (in fresh formalin solution, 24 hrs), and stored in formalin (10%). The period between initial anesthesia and fixation of the mesentery was kept below 60 minutes to minimize activation or de novo syntheses of MMPs during the tissue collection.

### Tissue Sections

Formalin fixed tissues were cut into 40 μm sections with a vibratome (Pelco Lancer Vibratome Series 1000).

### Serine Protease Immunohistochemistry

To determine on the tissue sections the immunolabel density and distribution of pancreatic serine proteases, pancreatic trypsin MoAb (D-1): sc-137077(Santa Cruz) primary antibody was used, followed by secondary antibodies (MP-7601 for anti-rabbit IgG; MP-7602 for anti-mouse IgG; ImmPRESS Excel staining kit peroxidase). Brown substrate color was used (ImmPACTTM DAB Substrate kit peroxidase, sk4105; and Vectorstain Elite ABC-HRP Kit, Vector® Laboratories). Sections without primary antibody served as controls. No counterstain was applied to facilitate quantitative label intensity measurements and since cellular and vascular structures are readily identified. The concentrations and exposure of primary and secondary antibodies applied to the sections were adjusted (24 hrs and according to protocol by Vector® Laboratories, respectively) to achieve full penetration of the antibodies into the skin tissue sections. All procedures were carried out under standardized conditions to permit quantitative comparison of label densities.

### Whole Mount Tissue Labeling

Small intestine: Full thickness tissue blocks of the wall of proximal jejunum (3 by 5 mm) were fixed from all sides in 10% formaldehyde. Pancreatic trypsin and amylase were detected with primary (MoAb (D-1) and MoAb (G-10), respectively), and secondary antibodies labeled with DAB (Peroxidase Substrate Kit, ab64238, ABCAM).

The mucin-containing mucus layer on the epithelial cells of the small intestine was stained using alcian blue (pH 2.5, kt 003; Diagnostic BioSystems, Pleasanton, CA) followed by a rinse in distilled water and mounted on a microscope slide (Vector Mount AQ Aqueous Mounting Medium, Vector Laboratories, Burlington, CA).

### Collagen Fragment Labeling

After removal of resin, 1 μm sections were labeled with biotin conjugated collagen hybridizing peptides (B-CHP) that bind unfolded collagen by triple helix formation and localize molecular level failure of collagen with high specificity.

Tissue sections were stained with B-CHP (stock solution, 150 mM; final applied solution 7.5. mM). The trimeric CHP are thermally dissociated to monomers before use (80° C. for 10 min), the hot CHP solution is quickly cooled to room temperature (by immersion into 4° C. water for 15 sec) and diluted and immediately applied to the section (dead time <1 min). In this way, most CHP peptides were expected to remain as active monomers during the staining process, based on kinetic studies on CHP triple helix folding. Sections were incubated overnight at room temperature, unbound B-CHP was removed by washing (3 times in 1 ml of 1×PBS for 30 min at room temperature). To visualize the B-CHP, the tissue sections were incubated with streptavidin peroxidase (sk-5704, Vector® Laboratories, according to manufacturer instructions) and then to a substrate (ImmPact AEC Substrate Kit Peroxidase; sk-4205, Vector Laboratories) at room temperature (for periods between 1 and 10 min depending on the tissue). The B-CHP label intensity on the sections was recorded by digital microscopy.

### Digital Image Analysis

Images of the immunolabel density were recorded from low power overviews of the tissue (10× objective, numerical aperture 0.25) under standard light conditions. Images were digitally analyzed to minimize operator error (NIH Image, 1.61, public domain software, spatial resolution of 640×480 pixel).

The intensity of the immune substrate label was measured after color extraction in form of light intensity (1 corresponding to white, 256 corresponding to black). The mean label density per group (with 3 animals per group) was determined from the average label density per animal (determined from 5 tissue sections/animal, 30 images/section).

### Example 2

### Loss of Mucin Coat in Rat Small Intestine

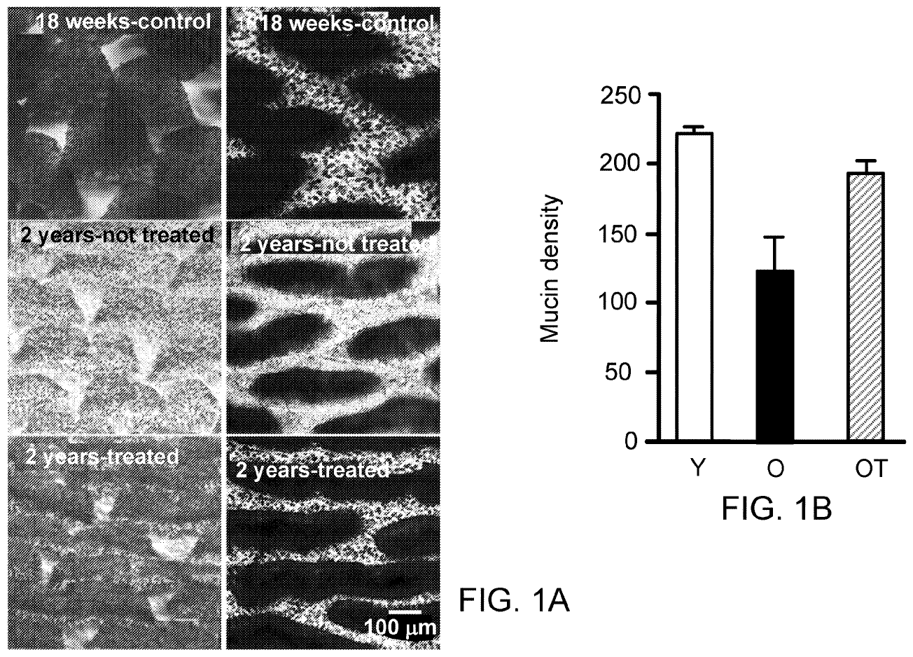

Immunohistochemistry was performed with alcian blue in rat intestine under the conditions described above for young, old, and old-treated animal groups, wherein it was demonstrated that a two-week-long blockade of the pancreatic serine protease trypsin restores the mucin coat in the small intestine of the old animals. En-face view images were taken of the inner lining of rat small intestine labeled for mucin, wherein the results show loss of the mucin coat in the rat small intestine of the old rats and restoration of the mucin coat in old rats after two-week oral treatment with a trypsin inhibitor (FIGS. 1A-1B).

### Example 3

### Pancreatic Trypsin and Amylase Accumulation in Rat Small Intestine

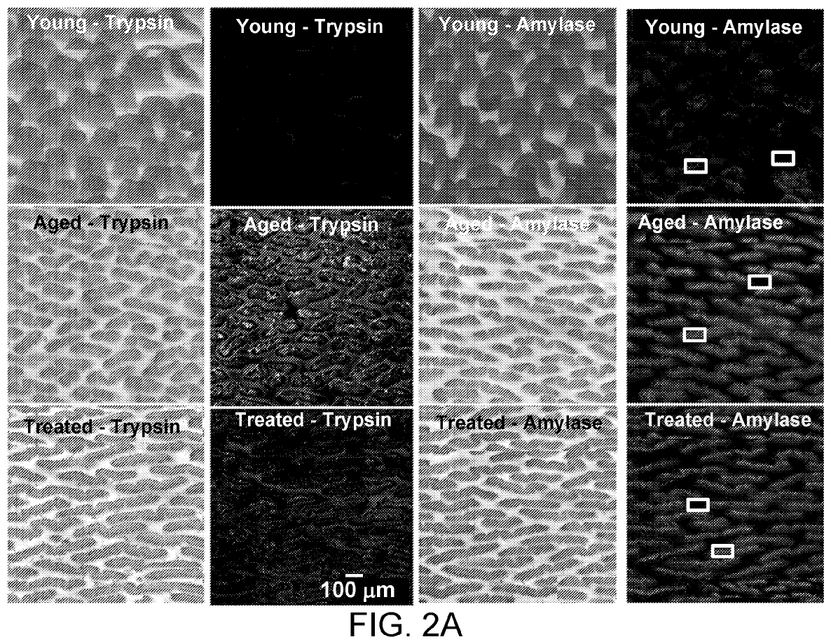

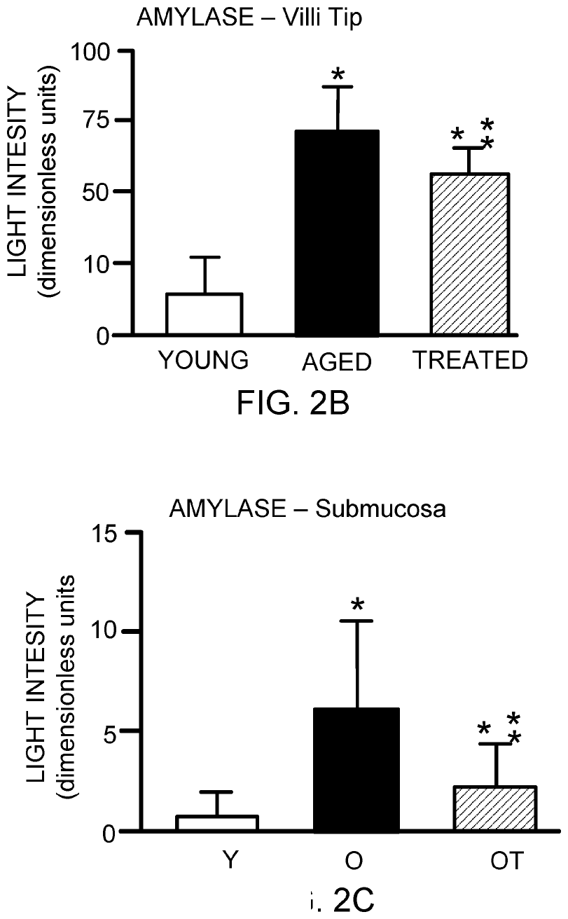

Immunohistochemistry was performed, and it was demonstrated that a two-week blockade of trypsin reduces pancreatic trypsin and amylase accumulation in the small intestine of old rats. En-face view images were taken of pancreatic trypsin (left two rows) and amylase (right two rows) label density co-labeled for mucin by alcian blue in the rat small intestine, wherein an increase in trypsin and amylase in the small intestine was observed in old rats compared to young rats, and this increase was attenuated by administration of oral tranexamic acid for two weeks (FIGS. 2A-2C).

### Example 4

### Pancreatic Trypsin Accumulation in Rat Skin Tissue

Immunohistochemistry was performed, and it was demonstrated that a two-week blockade of trypsin reduces pancreatic trypsin accumulation in skin tissue of old rats.

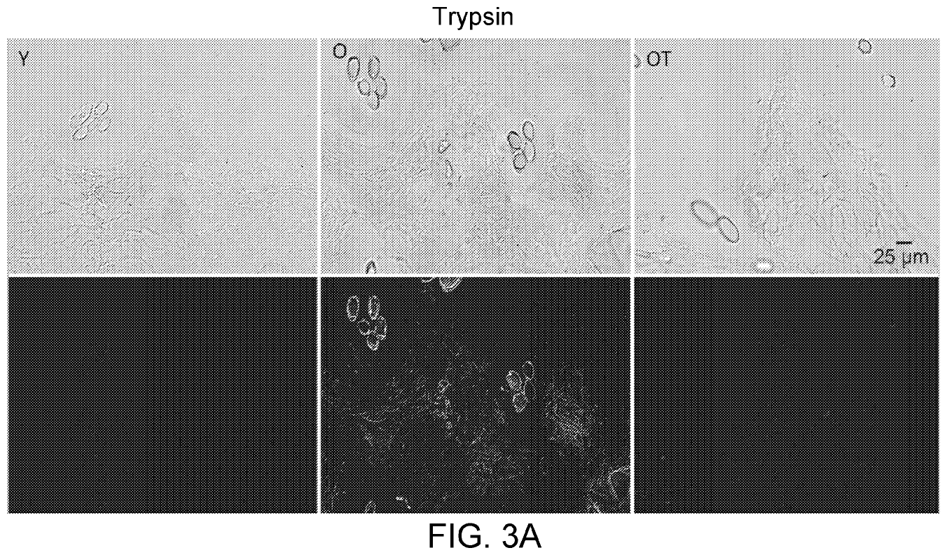

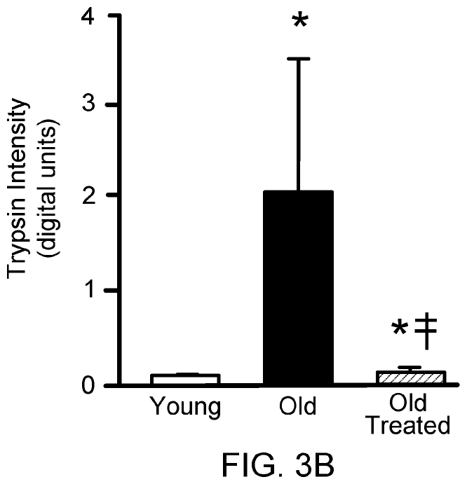

Immunohistochemistry was performed with a monoclonal antibody to pancreatic trypsin in young, old, and old-treated rat, wherein results showed that tranexamic acid for two weeks in old rats prevents trypsin accumulation in the skin tissue of old rats (FIGS. 3A-3B).

### Example 5

### Rat Skin Tissue Collagen Fragmentation

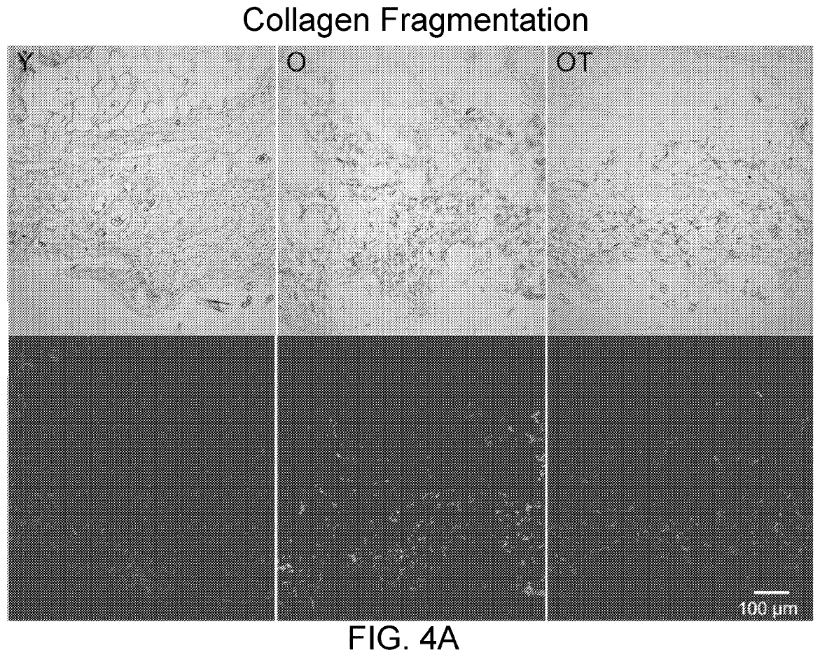

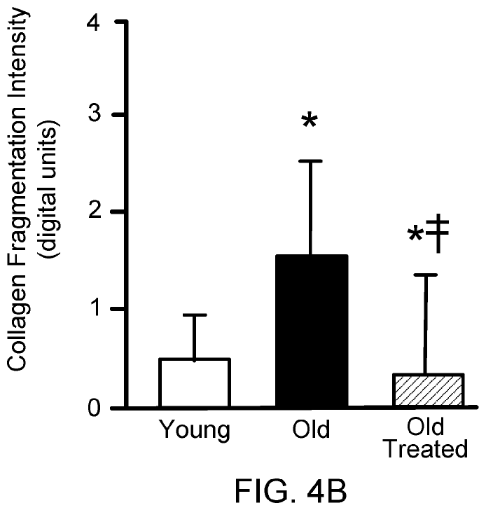

Immunohistochemistry was performed, and it was demonstrated that chronic blockade of trypsin reduces accumulation of digestive proteases in skin tissue. Rat skin tissue collagen fragmentation was observed in young mature, old, and old-treated rat. Results showed that tranexamic acid for two weeks in old rats prevents collagen fragmentation in the skin tissue of old rats (FIGS. 4A-4B), demonstrating the collagen fragmentation is likely caused by escaped digestive proteases.

### Example 6

### Exemplary Treatment of Potential Conditions in Subjects-Skin

A patient presents at the doctor with wrinkles and sagging skin. The doctor assesses the patient and determines that the patient likely has age-related skin changes. The doctor prescribes chronic administration of a composition disclosed herein for the treatment of age-related skin changes. The patient's age-related skin changes appear to stabilize.

What is claimed is:

1. A method of reversing accumulation of a pancreatic serine protease in skin tissue of a subject, comprising:
(a) selecting a subject having or at risk of accumulation of a pancreatic serine protease in the skin tissue; and
(b) administering a therapeutically effective amount of a serine protease inhibitor,
thereby reversing accumulation of the pancreatic serine protease in the skin tissue of the subject.

2. A method of reversing cellular damage in skin tissue of a subject, comprising:
(a) selecting a subject having or at risk of cellular damage to the skin tissue; and
(b) administering a therapeutically effective amount of a serine protease inhibitor,
thereby reversing cellular damage in the skin tissue of the subject.

3. A method of preserving extracellular matrix in the skin tissue of a subject, comprising:
(a) selecting a subject having or at risk of loss of extracellular matrix in the skin tissue; and
(b) administering a therapeutically effective amount of a serine protease inhibitor,
thereby preserving extracellular matrix in the skin tissue of the subject.

4. The method of any claim of the above, wherein the subject is at least 40 years old.

5. The method of any claim of the above, wherein the subject is at least 50 years old.

6. The method of any claim of the above, wherein the subject is at least 60 years old.

7. The method of any claim of the above, wherein the subject is not at risk of developing shock and/or septic shock.

8. The method of any claim of the above, wherein the subject does not have HIV.

9. The method of any claim of the above, wherein the pancreatic serine protease comprises at least one of a trypsin, a subtilisin, or combinations thereof.

10. The method of any claim of the above, wherein the pancreatic serine protease comprises at least one of a trypsin, an elastase, a chymotrypsin, or combinations thereof.

11. The method of any claim of the above, wherein the pancreatic serine protease comprises a trypsin.

12. The method of any claim of the above, wherein the serine protease inhibitor is a competitive inhibitor.

13. The method of any claim of the above, wherein the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soybean trypsin inhibitor, meprin inhibitors, setmelanotide, alpha-1-antitrypsin, and serpin.

14. The method of any claim of the above, wherein the serine protease inhibitor comprises FOY.

15. The method of any claim of the above, wherein the serine protease inhibitor comprises Futhane.

16. The method of any claim of the above, wherein the serine protease inhibitor comprises tranexamic acid.

17. The method of any claim of the above, wherein the therapeutically effective amount of the serine protease inhibitor is less than 10% of the pancreatic serine protease concentration of the GI tract.

18. The method of any claim of the above, wherein the therapeutically effective amount of the serine protease inhibitor is less than 10 μM.

19. The method of any claim of the above, wherein the therapeutically effective amount of the serine protease inhibitor is less than 5 μM.

20. The method of any claim of the above, wherein the serine protease inhibitor is orally administered.

21. The method of any claim of the above, wherein the serine protease inhibitor is administered as a liposomal encapsulation.

22. The method of any claim of the above, wherein the serine protease inhibitor is administered by a slow release device.

23. The method of any claim of the above, wherein the serine protease inhibitor comprises a composition that can be applied to the skin tissue.

24. The method of any claim of the above, wherein the serine protease inhibitor is administered superficially onto the skin tissue.

25. The method of any claim of the above, wherein the serine protease inhibitor is administered for more than 1 week.

26. The method of any claim of the above, wherein the serine protease inhibitor is administered for more than 2 weeks.

27. The method of any claim of the above, wherein the serine protease inhibitor is administered for more than 4 weeks.

28. A pharmaceutical composition for the treatment of aging or age-related conditions of skin tissue comprising a serine protease inhibitor.

29. The pharmaceutical composition of any claim of the above, wherein the serine protease inhibitor is a competitive inhibitor.

30. The pharmaceutical composition of any claim of the above, wherein the serine protease inhibitor is selected from the group consisting of nafamostat mesylate (Futhan), camostat mesilate (FOY 305), gabexate mesilate (FOY) or derivatives, serine protease inhibitor Kazal-type 1 (SPINK1), aprotinin, tranexamic acids, ulinastatin, granzyme A, granzyme B, UAMC-00050, 4-(2-minoethyl) benzenesulfonyl fluoride hydrochloride (AEBSF), soy bean trypsin inhibitor, meprin inhibitors, setmelanotide, alpha-1-antitrypsin, and serpin.

31. The pharmaceutical composition of any claim of the above, wherein the serine protease inhibitor comprises FOY.

32. The pharmaceutical composition of any claim of the above, wherein the serine protease inhibitor comprises Futhane 33. The pharmaceutical composition of any claim of the above, wherein the serine protease inhibitor comprises tranexamic acid.

34. The pharmaceutical composition of any claim of the above, wherein the serine protease inhibitor is administered at less than 10% of the subject's digestive enzyme activity.

35. The pharmaceutical composition of any claim of the above, wherein the serine protease inhibitor is less than 10 μM.

36. The pharmaceutical composition of any claim of the above, wherein the serine protease inhibitor is less than 5 μM.

37. The pharmaceutical composition of any claim of the above, wherein the serine protease inhibitor is orally administered.

38. The pharmaceutical composition of any claim of the above, wherein the serine protease inhibitor is administered as a liposomal encapsulation.

39. The pharmaceutical composition of any claim of the above, wherein the serine protease inhibitor is administered by a slow release device.

40. The pharmaceutical composition of any claim of the above, wherein the serine protease inhibitor comprises a composition that can be applied to the skin tissue.

41. The pharmaceutical composition of any claim of the above, wherein the serine protease inhibitor is administered superficially onto the skin tissue,

42. The pharmaceutical composition of any claim of the above, wherein the serine protease inhibitor is administered for more than 1 week.

43. The pharmaceutical composition of any claim of the above, wherein the serine protease inhibitor is administered for more than 2 weeks.

44. The pharmaceutical composition of any claim of the above, wherein the serine protease inhibitor is administered for more than 4 weeks.
